(19) United States
(12) Patent Application Publication
(10) Pub. No.: US 2025/0312143 A1
(43) Pub. Date: Oct. 9, 2025

(54) IMPLANTABLE BREAST PROSTHESIS

(71) Applicant: MEDICAL INNOVATION DEVELOPPEMENT, DARDILLY (FR)

(72) Inventor: Ali MOJALLAL, LYON (FR)

(73) Assignee: MEDICAL INNOVATION DEVELOPPEMENT, DARDILLY (FR)

(21) Appl. No.: 18/866,181

(22) PCT Filed: May 17, 2023
(86) PCT No.: PCT/FR2023/050713

§ 371 Date: Nov. 15, 2024

(30) Foreign Application Priority Data
May 17, 2022 (FR) 2204665

(51) Int. Cl. A61F 2/12

(52) U.S. Cl. CPC A61F 2/12; A61F 2250/0003; A61F 2250/0058

(57) ABSTRACT

An implantable breast prosthesis including a flexible inside skeleton defining a first filling chamber filled with a first fluid, and a flexible outside envelope defining a second filling chamber, composed of two communicating compartments surrounding the inside skeleton and filled with a second fluid of the hyaluronic acid type. The inside skeleton includes an inside base, an inside cover integrally secured to the inside base in such a way that the inner surfaces of the inside base and of the inside cover are facing each other, a bracing structure arranged between the inner surfaces of the inside base and of the inside cover and configured to space apart the inside cover and the inside base in such a way that the outer surface of the inside cover is convex.

### TECHNICAL FIELD

This disclosure relates to an implantable breast prosthesis.

### PRIOR ART

It is known to use breast prostheses that can be implanted in a patient's chest in order to achieve breast reconstruction, following an ablation or mastectomy in the context of cancer treatment or following necrosis resulting from infection or trauma. Breast prostheses can also be used for cosmetic purposes, to change the shape or volume of the breast.

Various types of breast prosthesis exist, which differ in particular by:
their shape, which for example can be anatomical, round, of the expandable prosthesis type or of the asymmetrical prosthesis type (left, right),
their outside envelope, which can be of polyurethane or silicone, for example,
the texture of their outside envelope, which for example can be smooth, microtextured or macrotextured,
by the product filling the outside envelope, which for example can be silicone gel, physiological saline solution, or hydrogel.

In addition to the risks associated with the appearance of periprosthetic capsule, folds, rippling or rotation, inflammation or infection, known breast prostheses primarily pose a risk of rupture or deflation resulting in leakage of the filling product. When silicone gel is involved, such leakage constitutes a real danger for the patient, given the local and general inflammation, the invasion of the lymph nodes, and the siliconoma formation which can all result.

There is therefore a need for a breast prosthesis which poses reduced risks for the patient.

Patient comfort and a breast restoration that meets the patient's expectations as closely as possible, particularly in terms of aesthetics and feel, are also essential specifications.

### SUMMARY

The present disclosure is in response to this need.

An implantable breast prosthesis is proposed, comprising:
a flexible inside skeleton defining a first filling chamber filled with a first fluid, this inside skeleton comprising:
an inside base having opposite inner and outer surfaces,
an inside cover having inner and outer surfaces and integrally secured to the inside base in such a way that the inner surfaces of the inside base and of the inside cover are facing each other,
a bracing structure arranged between the inner surfaces of the inside base and of the inside cover and configured to space apart the inside cover and the inside base in such a way that the outer surface of the inside cover is convex,
a flexible outside envelope defining a second filling chamber filled with a second fluid, this second filling chamber surrounding the inside skeleton.

The breast prosthesis according to the invention thus provides a bracing structure ensuring that a shape, resistance, and volume are maintained regardless of the first and second fluids filling the first and second filling chambers. According to some specific arrangements, the bracing structure allows eliminating the use of silicone gel, which is a cause of known major problems.

One of the advantages of this prosthesis is also its lightness.

Another positive point to be credited to this prosthesis is the uniform transfer of force at the surface (second chamber), during palpation.

According to some specific arrangements, the breast prosthesis according to the invention allows reducing the surgical trauma associated with its placement in the retromuscular position. The breast prosthesis can in particular allow prepectoral placement with a natural result, thus avoiding a partial section or recession of the pectoral muscle.

The two-part design of the breast prosthesis, namely with an inside skeleton and an outside envelope, also makes it possible, according to some specific arrangements, to:
provide a safer texture of the outside envelope, which is now implicated in the pathophysiology of anaplastic lymphomas, and in chronic inflammatory reactions,
have a safer installation technique, eliminating all risk of rupture (trauma to the implant) during placement,
secure the position of the breast prosthesis, to avoid secondary displacements and rotations and the subsequent surgical interventions to remedy them; the breast prosthesis can indeed have one or more attachment elements securing its position, replacing the attachments via periprosthetic inflammation of the polyurethane breast prostheses.

Advantageously:
the inside base has an outer edge extending around a base axis (A);
and the bracing structure comprises at least one flexible bracing wall having first and second opposite edges respectively secured to the inner surfaces of the inside base and of the inside cover.

In a first embodiment shown in FIGS. 1 to 3, the bracing structure is a honeycomb structure (cells of hexagonal or circular cross-section, for example).

In a second embodiment shown in FIGS. 4 to 6, the first edge and the second edge are aligned according to the base axis (A) and the first edge is preferably substantially perpendicular to the base axis (A).

In a third embodiment not shown in the figures, the wall(s) of the bracing structure has (have) an annular shape with an axis (A).

For some of the walls of the honeycomb structure of the first embodiment and for at least part of the walls, preferably for all the walls, in the second embodiment, the second edge can be aligned with the first edge according to the base axis (A).

In these three embodiments, and others, the bracing structure comprises an arrangement of bracing walls, configured to define a plurality of compartments in the first filling chamber.

In some of these embodiments, in particular in the first and second embodiment, the compartments respectively have compartment axes (B) parallel to the base axis (A).

According to an alternative embodiment, the bracing walls of the structure of the same name may have orifices which place the compartments of the first filling chamber in communication. This allows some circulation of the first filling fluid in this first chamber, between some or all of the compartments.

According to one noteworthy feature of the invention, the bracing structure has a vertex centered on the base axis.

According to some advantageous arrangements of the invention,
the outside envelope of the prosthesis has an inner surface oriented towards the second filling chamber and an outer surface opposite to the inner surface;
the inside skeleton has a peripheral skirt integrally secured to the inner surface of the outside envelope in such a way that the second filling chamber has:
a first compartment between the inner surface of the outside envelope, the outer surface of the inside cover, and the peripheral skirt,
and a second compartment between the inner surface of the outside envelope, the outer surface of the inside base, and the peripheral skirt.

In accordance with a preferred feature of the invention, the first and second compartments of the second chamber are in fluid communication, preferably by means of holes provided through the peripheral skirt, these holes more preferably being evenly distributed.

This feature provides a buffer zone for vibrations, which greatly contributes to the comfort of the prosthesis.

Preferably, the outside envelope of the prosthesis comprises:
an outside base having an inner surface facing the outer surface of the inside base,
and an outside cover having inner and outer surfaces;
   the outside base and the outside cover preferably being configured so that the inside and outside bases are parallel to each other.

The outside base, the peripheral skirt, and the inside base thus define the second compartment of the second filling chamber.

According to one possibility, the outside base can be a different part than the outside cover and thus be attached and integrally secured to the outside cover and to the peripheral skirt after filling the two compartments of the second filling chamber with the second filling fluid.

The inside skeleton and the outside envelope of the prosthesis are preferably made of a natural or synthetic elastomeric material of a Shore A hardness that is preferably between 10 and 80, more preferably between 15 and 50, and even more preferably between 25 and 35.

This elastomeric material is advantageously selected from the group comprising—ideally consisting of—the following materials:
silicones, in particular “Liquid Silicon Rubber” (LSR) silicones or “Heat Cured Rubber or High Consistency Rubber” (HCR) silicones; in particular silicones comprising polydimethylsiloxanes (PDMS);
plastomers, in particular polydimethylsiloxane (PDMS) and polymethyl methacrylate (PMMA) block copolymers;
copolymers comprising PLA (PLA: polylactic acid), in particular PLA-b-PEG (PEG: polyethylene glycol);
and mixtures of these materials.

The first and second filling fluids can be different from each other, the first fluid being gaseous at a temperature greater than or equal to 30° C. and at atmospheric pressure, the second fluid being liquid at a temperature greater than or equal to 30° C. and at atmospheric pressure.

The first fluid can comprise air, preferably at a pressure equal to atmospheric pressure.

The second fluid can comprise a solution having a viscosity of between 100 and 500Pa·s, preferably between 200 and 300 Pa·s. This viscosity is measured according to the method described in “2.2.10. VISCOSITY-ROTATING VISCOMETER METHOD” on pages 28 & 29 of “EUROPEAN PHARMACOPEIA 8.0”, making use of a “CONCENTRIC CYLINDER VISCOMETER ABSOLUTE VISCOMETER” and implementing a “Couette type viscometer” (non-Newtonian fluid) as shown in FIGS. 2.2.10.-1 and 2.2.10.-2, at an average shear rate of 1 s 1, varying between 0.1 s 1 and 10 s 1, at ambient temperature and ambient atmospheric pressure.

The second fluid can comprise a solution of hyaluronic acid, preferably at a concentration of between 1% by weight and 5% by weight, and even more preferably between 2% by weight and 4% by weight.

The outside envelope can advantageously include at least one attachment element.

According to another aspect, a method for manufacturing the breast prosthesis as defined above is proposed, by molding and/or 3D printing the prosthesis in one piece or the component elements of the prosthesis in several pieces, preferably by molding and/or 3D printing the flexible inside skeleton and/or the flexible outside envelope in one piece or several pieces; even more preferably by molding and/or 3D printing the bracing structure and/or the inside cover, and/or the peripheral skirt, in one piece or in several pieces;
said method further comprising:
advantageously, filling the first and/or the second filling chamber with the first and the second filling fluid respectively;
and, possibly, assembling the component elements of the prosthesis.

According to one advantageous arrangement of the invention, some or all of the component elements of the prosthesis defining at least one of the two filling chambers are subjected to a sealing treatment, preferably by application of a coating, preferably a polymer coating, and more preferably a parylene coating.

### BRIEF DESCRIPTION OF DRAWINGS

Other features, details, and advantages will become apparent upon reading the detailed description below, and upon analyzing the appended drawings, in which:

FIG. 1 is a perspective-view representation of an implantable breast prosthesis according to a first embodiment,

FIG. 2 is a top-view representation of the breast prosthesis of FIG. 1,

FIG. 3 is a cross-sectional representation, along the orientation denoted III-III in FIG. 2, of the breast prosthesis of FIG. 1,

FIG. 4 is a perspective-view representation of an implantable breast prosthesis according to a second embodiment,

FIG. 5 is a top-view representation of the breast prosthesis of FIG. 4,

FIG. 6 is a cross-sectional representation, along the orientation denoted VI-VI in FIG. 5, of the breast prosthesis of FIG. 4.

### DESCRIPTION OF EMBODIMENTS

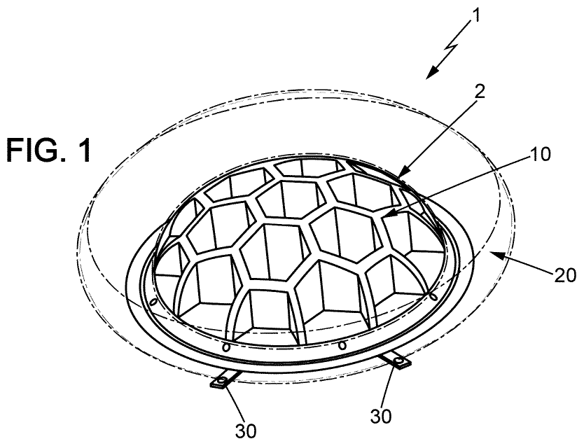

FIGS. 1 to 3 represent a first embodiment of a breast prosthesis 1 intended to be implanted in the chest of a patient, in a context of breast reconstruction surgery or cosmetic surgery.

Breast prosthesis 1 comprises a flexible inside skeleton 2 and a flexible outside envelope 20, both made of a deformable biocompatible material, such as a silicone elastomer with a shore A hardness for example that is equal to 30.

The material is preferably radio-transparent, so that it does not interfere with reading mammograms and breast ultrasounds.

Inside skeleton 2 comprises an inside base 4 and an inside cover 5 assembled to each other in a sealed manner in order to define a first filling chamber 3. Said chamber is filled or is to be filled with the first filling fluid prior to assembly. According to one alternative, inside skeleton 2 could be made in a single piece, the filling of the first chamber with the first fluid then preferably taking place simultaneously with the manufacture of inside skeleton 2.

Inside base 4 has opposite inner 4a and outer 4b surfaces and an outer edge which, in the first embodiment shown, extends around a base axis A while being centered on base axis A. In particular, the outer edge is circular.

Inside cover 5 is integral with inside base 4 (for example after joining by gluing or welding), in such a way that an inner surface 5a of inside cover 5 is arranged facing inner surface 4a of inside base 4. In particular, inner surface 4a of inside base 4 is integral with inner surface 5a of inside cover 5 (for example after joining by gluing or welding), at a distance from a peripheral edge 6 thereof, in such a way that inside cover 5 comprises a peripheral skirt 7 between its peripheral edge 6 and the outer edge of inside base 4.

A bracing structure 10 is interposed between inner surfaces 4a, 5a of inside base 4 and of inside cover 5, to space apart inside cover 5 and inside base 4, in such a way that:
inside base 4 can be generally flat when it rests on a flat support surface, in the absence of external forces,
and an outer surface 5b of inside cover 5, opposite to inner surface 5a, is convex.

Bracing structure 10 comprises an arrangement of flexible bracing walls 11 each having first 12 and second 13 opposite edges, respectively secured to inner surfaces 4a, 5a of inside base 4 and of inside cover 5. In order to ensure that inside skeleton 2 is maintained in the desired shape, namely a flat inside base 4 and a convex inside cover 5, first edge 12 of each of bracing walls 11 extends perpendicularly to base axis A and second edge 13 is aligned with first edge 12 according to base axis A. In addition, the second edges 13 of bracing walls 11 have curvatures adapted to obtain convexity of outer surface 5b of inside cover 5. In the first embodiment shown, bracing structure 10 has a vertex centered on base axis A, giving inside skeleton 2 a dome shape.

Bracing walls 11 are arranged to define a plurality of compartments 14 respectively having compartment axes B which are parallel to base axis A. In the first embodiment shown in FIGS. 1 to 3, the arrangement of bracing walls 11 forms a honeycomb type of three-dimensional bracing structure 10, its cells in this example having a hexagonal shape in a cross-sectional plane perpendicular to base axis A. These cells are compartments 14, preferably sealed relative to each other.

Outside envelope 20 of prosthesis 1 comprises an outside base 22 and an outside cover 23 which are one piece or are integrally secured to each other in a sealed manner to define a second filling chamber 21 surrounding inside skeleton 2 and filled with the second filling fluid.

Peripheral skirt 7 of inside skeleton 2 is integrally secured to an inner surface 20a of outside envelope 20, such that outer surface 4b of inside base 4 faces an inner surface 22a of outside base 22. Second filling chamber 21 then has a first compartment 21a between an inner surface 23a of outside cover 23 of outside envelope 20, outer surface 5b of inside cover 5, and peripheral skirt 7, and a second compartment 21b between inner surface 22a of outside base 22 of outside envelope 20, outer surface 4b of inside base 4, and peripheral skirt 7.

First filling chamber 3 of inside skeleton 2 is filled with a first fluid and second filling chamber 21 of outside envelope 20 is filled with a second fluid. First 21a and second 21b compartments communicate with each other by means of holes 25, for example evenly distributed, formed in peripheral skirt 7 of inside skeleton 2. The second filling fluid can thus circulate between these two compartments 21a and 21b. Outside base 22 and outside cover 23 are configured so that inside 4 and outside 22 bases are parallel to each other at rest and when the prosthesis filled with the two fluids is placed on a flat support. In this rest position, outer surface 23b of outside cover 23 of outside envelope 20 is convex.

The first fluid is gaseous. It is for example air, preferably at a pressure equal to atmospheric pressure.

The second fluid is, for example, an aqueous solution of hyaluronic acid at a concentration of about 3% by weight.

To improve the retention in position of breast prosthesis 1, the prosthesis may comprise, preferably on the outside envelope, one or more attachment elements, for example in the form of attachment tabs.

Breast prosthesis 1 thus produced has mechanical properties such as flexibility, behavior, resistance, and density, which give it satisfactory firmness aesthetically and to the touch.

Bracing structure 10 can have different sizes of cells, according to whether their location is central or peripheral relative to base axis A for example, in order to create an inside skeleton 2 that is more or less dense, providing varying suppleness on palpation.

Breast prosthesis 1 is also compressible and deformable so that it can be partially crushed and flattened for implantation through an incision of between 2 cm and 10 cm, preferably between 3 cm and 6 cm, and even more preferably about 4 cm.

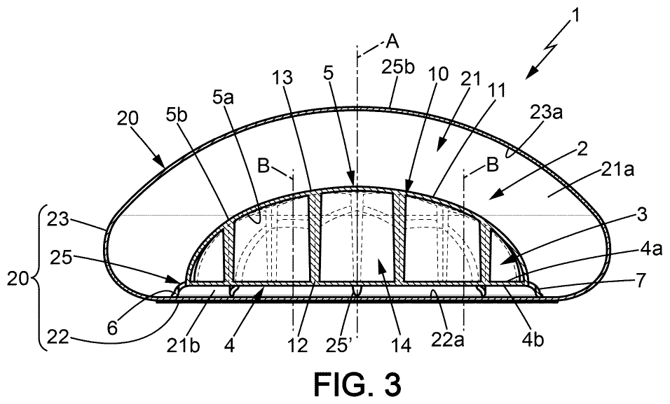

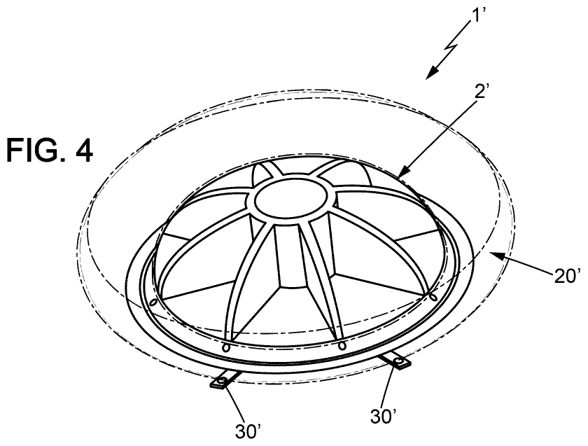

In relation to FIGS. 3 to 6, a second embodiment of a breast prosthesis 1′ is described.

In this second embodiment, breast prosthesis 1′ differs from the first embodiment only in the arrangement of flexible bracing walls 11′ of bracing structure 10′ of inside skeleton 2′. The other features, unchanged from the first embodiment, are not described again here. One will therefore refer to the description given above for more details.

Bracing walls 11′, of which there are eight in the figures, extend radially relative to base axis A, from a hollow cylindrical trunk 26′ centered on base axis A. This arrangement defines a central compartment 14′ and eight peripheral compartments 14′ around central compartment 14′.

Alternatively, hollow cylindrical trunk 26′ can be replaced by a solid trunk of variable diameter. The minimum diameter can correspond to the dimension where walls 11′ meet around base axis A.

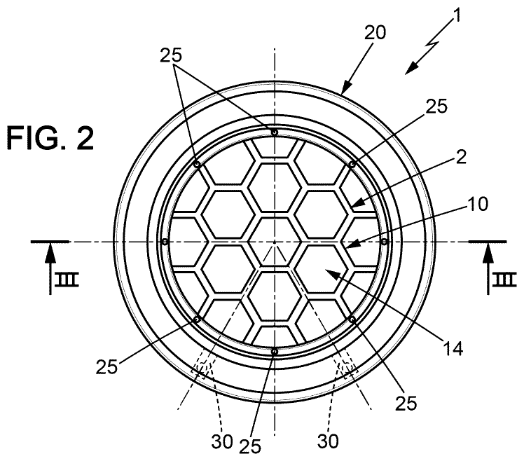

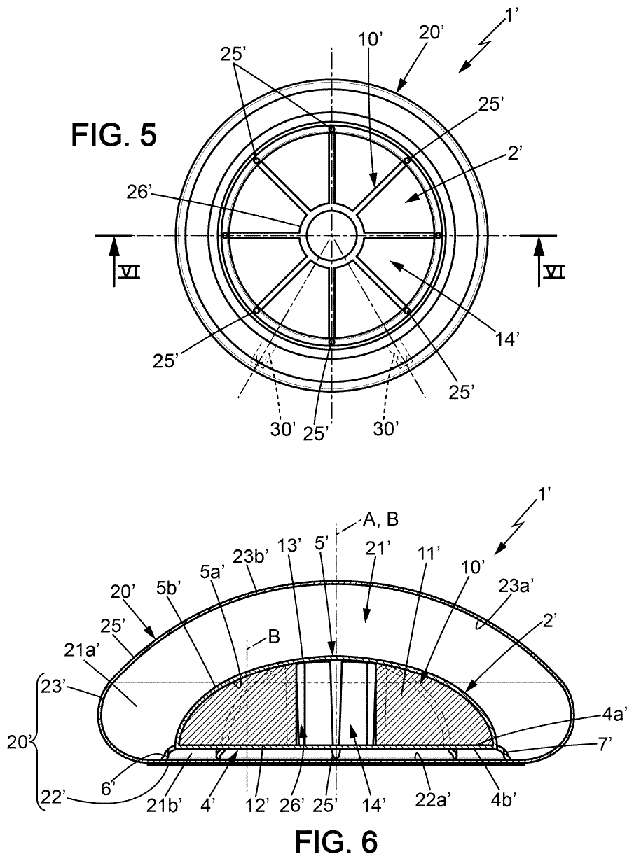

According to a preferred feature, breast prosthesis 1,1′ comprises two attachment tabs 30,30′ each having an eyelet enabling attachment, e.g. by sewing, of the prosthesis after placement. As shown in FIGS. 2 and 5, two attachment tabs 30,30′ are symmetrical relative to the sagittal diametral plane of the prosthesis, intended to be substantially parallel to the sagittal plane of the patient after placement of prosthesis 1,1′. The angle formed by each attachment tab 30, 30′ relative to the sagittal diametral plane of the prosthesis is preferably between 45 and 15 degrees, and more preferably about 30°.

Alternatively, any other arrangement of one or more flexible bracing walls 11,11′ spacing apart the inside cover 5 and the inside base 4 while providing convexity on inside cover 5 could be provided.

Thus, the bracing walls could be one or more rings, in particular circular, oval, or elliptical, continuous or discontinuous, concentric or not concentric with axis A, or any other wall shapes.

With regard to the method for manufacturing breast prosthesis 1, 1′, it concerns known molding or 3D printing technologies, using elastomers, for example silicones having a Shore A hardness of around 30 after crosslinking/curing. Molding is understood in particular to mean injection molding, pressing, stamping, etc.

The various component elements of breast prosthesis 1,1′ can be manufactured separately, as one unit or as several subunits. When breast prosthesis 1,1′ is not produced as a single unit, the manufacturing method comprises operations for assembling the various component elements or sub-units, by welding and/or gluing.

To seal or to improve the seal of the various walls separating compartments 14, 14′, 21a, 21a′, 21b, 21b′, a coating treatment by a polymer, for example parylene, is implemented.

The manufacture of prosthesis 1,1′ preferably includes filling the first filling chamber with a first gaseous fluid, such as air at atmospheric pressure, and filling the second filling chamber composed of the two compartments 21a, 21a′, 21b, 21b′ enclosing inside cover 5,5′, with a second fluid formed of a liquid, for example an aqueous solution of hyaluronic acid at 2 or 3% by weight.

1-11. (canceled)

12. Implantable breast prosthesis comprising:
a flexible inside skeleton defining a first filling chamber filled with a first fluid, this inside skeleton comprising:
an inside base having opposite inner and outer surfaces, an inside cover having inner and outer surfaces and integrally secured to the inside base
in such a way that the inner surfaces of the inside base and of the inside cover are facing each other,
a bracing structure arranged between the inner surfaces of the inside base and of the inside cover and configured to space apart the inside cover and the inside base in such a way that the outer surface of the inside cover is convex, and
a flexible outside envelope defining a second filling chamber filled with a second fluid, this second filling chamber surrounding the inside skeleton.

13. The breast prosthesis according to claim 12, wherein:
the inside base has an outer edge extending around a base axis (A); and
the bracing structure comprises at least one flexible bracing wall having first and second opposite edges respectively secured to the inner surfaces of the inside base and of the inside cover.

14. The breast prosthesis according to claim 12, wherein the bracing structure comprises an arrangement of bracing walls, configured to define a plurality of compartments in the first filling chamber.

15. The breast prosthesis according to claim 12, wherein the bracing structure has a vertex centered on the base axis (A).

16. The breast prosthesis according to claim 12, wherein
the outside envelope has an inner surface oriented towards the second filling chamber and an outer surface opposite to the inner surface;
the inside skeleton has a peripheral skirt integrally secured to the inner surface of the outside envelope in such a way that the second filling chamber has:
a first compartment between the inner surface of the outside envelope, the outer surface of the inside cover, and the peripheral skirt, and a second compartment between the inner surface of the outside envelope, the outer surface of the inside base, and the peripheral skirt.

17. The breast prosthesis according to claim 16, wherein the first and second compartments of the second filling chamber are in fluid communication, preferably by means of holes provided through the peripheral skirt, these holes more preferably being evenly distributed.

18. The breast prosthesis according to claim 12, further comprising, preferably on the outside envelope, one or more attachment elements or tabs.

19. The breast prosthesis according to claim 12, wherein the first and second fluids are different from each other, the first fluid being gaseous at a temperature greater than or equal to 30° C. and at atmospheric pressure, the second fluid being liquid at a temperature greater than or equal to 30°° C. and at atmospheric pressure.

20. The breast prosthesis according to claim 19, wherein the first fluid comprises air, preferably at a pressure equal to atmospheric pressure.

21. The breast prosthesis according to claim 20, wherein the second fluid is a solution of hyaluronic acid, preferably at a concentration of between 1% by weight and 5% by weight, and even more preferably between 2% by weight and 4% by weight.

22. A method for manufacturing the breast prosthesis according to claim 12, by molding and/or 3D printing the prosthesis in one piece or the component elements of the prosthesis in several pieces, preferably by molding and/or 3D printing the flexible inside skeleton and/or the flexible outside envelope in one piece or in several pieces; even more preferably by molding and/or 3D printing the bracing structure and/or the inside cover, and/or the peripheral skirt, in one piece or in several pieces;
said method further comprising:
advantageously, filling the first and/or the second filling chamber with the first and the second filling fluid respectively; and
possibly, assembling the component elements of the prosthesis.
